(19) United States
(12) Patent Application Publication
(10) Pub. No.: US 2008/0081021 A1
(43) Pub. Date: Apr. 3, 2008

(54) Manganese-enhanced magnetic resonance imaging of neurons using electrical stimulation

(76) Inventors: Mehmet Bilgen, Kansas City, KS (US); William M. Brooks, Kansas City, MO (US); Paul D. Cheney, Olathe, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/540,264

(22) Filed: Sep. 29, 2006

(51) Int. Cl. A61K 49/10

(52) U.S. Cl. 424/9.36

(57) ABSTRACT

A method for improving uptake of contrast agents, such as manganese-based contrast agents, is neuronal imaging of areas such as the spinal cord and cortical spinal tract with magnetic resonance imaging are provided. Electrical stimulation is applied to the subject in order to increase uptake of the contrast agent in to the neuron, resulting in an improved image.

### STATEMENT REGARDING FEDERALLY SPONSORED RESEARCH OR DEVELOPMENT

The present invention was supported in part by NIH Grant NS051825 and the government may have certain rights in the invention.

### CROSS-REFERENCE TO RELATED APPLICATIONS

Not applicable.

### BACKGROUND OF THE INVENTION

1. Field of the Invention

The present invention relates to methods for enhancing neuroimaging using contrast agents, such those involving magnetic resonance imaging (“MRI”). More specifically, improved methods of imaging the neuronal tracts, such as those in the cortical spinal tract and spinal cord using manganese-enhanced MRI are disclosed.

2. Description of Related Art

Various neuroimaging techniques which employ contrast agents are known in the art. Such techniques include computer tomography (“CT”), computer axial tomography (“CAT”), positron emission tomography (“PET”), single photon emission computed tomography (“SPECT”), and MRI. In particular, MRI is well established as a medical diagnostic tool. The ability of the technique to generate high quality images and to differentiate between soft tissues without requiring the patient to be exposed to ionizing radiation has contributed to this success.

Although MRI can be performed without using added contrast agents, various materials having paramagnetic, superparamagnetic or ferromagnetic properties, are frequently used to enhance imaging. Species with unpaired electrons, such as the paramagnetic transition and lanthanide metal ions, are frequently employed. Often, these contrast agents are associated with chelators in order to help avoid toxic effects. Many currently used well-known paramagnetic agents include ferric ammonium citrate, gadolinium-DTPA, chromium-DTPA, chromium-EDTA, maganese-DTPA, manganese-EDTA, manganese chloride, iron sulfate and mixtures thereof. Exemplary contrast agents are disclosed in Brechbiel, U.S. Pat. No. 6,852,842, and Mulder et al., Lipid-based nanoparticles for contrast-enhanced MRI and molecular imaging, NMR Biomed. 19(1):142-64 (2006), which are incorporated by reference. Further, isotopes of the contrast agents are often used for many imaging techniques. For example, 54 Mn may be used in SPECT or PET imaging of neuronal tissues, and it has been reported to be transported axonally. See Gallez et al., Accumulation of manganese in the brain of mice after intravenous injection of manganese-based contrast agents, Chem Res Toxicol. 10(4):360-3 (1997), Sloot et al., Axonal transport of manganese and its relevance to selective neurotoxicity in the rat basal ganglia, Brain Res. September 19;657(1-2):124-32 (1994).

One contrast agent for use in brain imaging that has received attention in recent years is manganese ion. As a biological calcium ion analog, manganese ion is known to enter neurons via L-type voltage gated calcium channels. Researchers have also demonstrated that manganese undergoes microtubule-associated axonal transport. See Sloot and Gramsbergen, Axonal transport of manganese and its relevance to selective neurotoxicity in the rat basal ganglia, Brain Res. 19 657(1-2):124-32 (September 1994).

Most of the manganese-enhanced MRI involving neuronal circuitry has involved the brain, and only a few attempts have been made to image the spinal cord. See Allegrini and Wiessner, Three-dimensional MRI of cerebral projections in rat brain in vivo after intracortical injection of MnCl₂, NMR Biomed. 16(5):252-6 (August 2003); Aoki et al., In vivo detection of neuroarchitecture in the rodent brain using manganese-enhanced MRI, Neuroimage 22(3): 1046-59 (July 2004); Leergaard et al., In vivo tracing of major rat brain pathways using manganese-enhanced magnetic resonance imaging and three-dimensional digital atlasing, Neuroimage 20(3):1591-600 (November 2003). Unfortunately, in these previous studies, manganese labeling of the cortical spinal tract weakened as the tract approached the pyramidal decussation, before reaching the cervical spinal cord. Thus, there remains a need to improve manganese-enhanced MRI of neuronal tissue in such areas as the spinal cord.

### BRIEF SUMMARY OF THE INVENTION

The present invention is directed to a method for improving imaging of neurons in areas such as the spinal cord and the brain. In a preferred embodiment, electrical stimulation is used to improve manganese-enhanced MRI. It is theorized that electrical stimulation activates voltage-gated calcium channels, which in turn increases manganese ion uptake and leads to improved manganese-enhanced MRI.

Thus, in one aspect, the present invention is directed to a method for enhancing imaging of a neuronal tissue, organ, or system in a mammal comprising administering a diagnostically effective amount of a contrast agent, and stimulating the neurons in order to increase uptake of the contrast agent into the neurons. The imaging is preferably magnetic resonance imaging, and the stimulating step comprises applying electrical stimulation to the mammal. In still another aspect, the mammalian neuronal tissue that is imaged comprises the mammal's cortical spinal tract or spinal cord.

In another aspect, calcium channels are opened in order to increase the uptake of the contrast agent into the cell. Electrical stimulation may be applied to the motor cortex in order to open the calcium channels. Further, a calcium channel agonist may be administered to the mammal. Other indirect pathways for opening the calcium channels are also contemplated to improve contrast agent uptake by the cell.

In one aspect, the contrast agent is a paramagnetic metal. Preferred contrast agents are those derived from manganese, for example, in either a salt or chelated form.

In still another aspect, the contrast agent is administered to the patient intracortically or intrathecally.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 shows the projection of the cortical spinal tract in the rat central nervous system. The arrowhead denotes the site of intracortical manganese injection in the brain. In vivo data in (panels A-D) were obtained after 24 hour of injection and electrical stimulation of the motor cortex. Panel (A) is a sagittal MIP image constructed over a thickness of 4 mm. Panel (B) is a coronal MIP image constructed over a thickness of 8 mm. Panel (C) is manganese enhanced MRIs showing manganese enhancement of the cortical spinal tract in rat brain in axial views. Panel (D) is manganese enhanced MRIs in axial views showing the manganese enhancement in the pyramidal tract at the medulla, in the pyramidal decussation and dorsal fasiculus in spinal cord. Panel (E) is a cortical spinal tract-tracing image similar to the one in panel (A) acquired after 24 hours of injection, but without the electrical stimulation of the motor cortex. SI—primary somatosensory cortex, ic—internal capsule, thal—thalamus, cp—cerebral peduncle and py—pyramidal tract.

FIG. 2 shows the in vivo axial spin-echo images of a rat spinal cord acquired at T_{R} values of 0.05 (top left), 0.1, 0.25, 0.5, 0.75, 2.0, 3.5 and 6.0 s (bottom right), when T_{E}=12 ms. The arrows point to the hyperintensities overlaying the anatomical location of the cortical spinal tract in spinal cord.

FIG. 3 is a T₁-map of a rat spinal cord computed from the image series in FIG. 2. The arrow points to the cortical spinal tract.

FIG. 4 is a three-dimensional visualization of the rat central nervous system and projection of the cortical spinal tract pathway. The data were acquired using 3D GE-manganese enhanced MRIs after 24 hours of intracortical injection of manganese and electrical stimulation of the motor cortex. The red color denotes the manganese-enhanced labeling of the cortical spinal tract in brain and spinal cord.

FIG. 5 shows the in vivo visualization of the rat central nervous system and cross sectional views of the cortical spinal tract pathway in the sagittal and axial planes. This data were acquired using 3D GE-manganese enhanced MRIs (panels (a) and (b)) and IR-manganese enhanced MRIs (panels (c) and (d)). The arrow labeled “SCI” points to the lesion at the T4 level. The thin rectangles overlaid on the sagittal images represent the slice orientation for the axial images. The arrowhead denotes the site of the manganese injection in the brain, where the signal hypointensity is due to the presence of high local concentration of manganese. SI—primary somatosensory cortex, ic—internal capsule, thal—thalamus, cp—cerebral peduncle and py—pyramidal tract.

FIG. 6 panels (a) and (b) show the ex vivo spine echo images showing the injury (arrow head) in an excised spinal cord but intact spine from rostral (left) to caudal (right) direction in sagittal planes. Panels (c), (d), and (e) show the ex vivo anatomical axial spine echo images of the spinal cord from the three slice locations (panel (c)—rostral to injury, panel (d)—injury epicenter and panel (e)—caudal to injury) depicted by the thin rectangles overlaid on the sagittal image in panel (b).

FIG. 7 shows the ex vivo IR-manganese enhanced MRIs from the same slice orientations as those images in FIG. 6, panels (c), (d), and (e). Panel (a) shows the manganese-enhanced cortical spinal tract rostral to the injury. Panel (b) shows the manganese-enhancement at the epicenter of the injury. Panel (c) shows the manganese labeling of the cortical spinal tract caudal to the injury. The partial signal enhancement, depicted by the arrowhead in panel (b), is likely to represent a portion of the cortical spinal tract that is populated with intact fibers. By continuously projecting through the injury site, these intact fibers transport manganese from rostral to caudal sections as indicated by the presence of focal signal enhancement in panel (c).

FIG. 8 shows a cigar-shaped ellipsoidal representation of the principal eigenvectors estimated from the DTI measurements. The eigenvector estimates from the Manganese-labeled regions were only plotted on backgrounds that are the same as those rostral, epicenter and caudal images in FIG. 7. Therefore, the density of the vectors is associated with the size of the manganese-enhancement. Vector directions are all aligned along the cord in all the three images. The direction of the alignment is consistent with the anatomical orientation of the descending neuronal fibers in the cortical spinal tract. The DTI data acquisition included first the baseline image, followed by the diffusion-weighted images obtained with applying diffusion sensitizing gradients along the directions (110,101,011,−110,−101,0−11). Diffusion weighting was achieved using gradient strength=80 mT/m, width (δ)=6.5 ms and separation (Δ)=11 ms to produce b-value of b=342 s/mm². Other parameters were TR/TE=2500/26 ms, FOV=10×10 mm², acquisition matrix=128×128, slice thickness=2 mm and NEX=2.

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

### DETAILED DESCRIPTION OF PREFERRED EMBODIMENT

The present invention is directed to a method for enhancing imaging of a mammalian neuronal tissue, organ or system. Improved neuronal imaging is achieved by increasing the uptake of the contrast agent into the neuron.

A diagnostically effective amount of the contrast agent is administered to the subject. The term “diagnostically effective” refers to an amount of a contrast agent sufficient to increase the signal-to-noise ratio for the imaging technique of the tissue in question.

Various contrast agents are used in neuronal imaging. Preferred contrast agents are nontoxic and are characterized as being taken into the cell and transported axonally. The most preferred contrast agent is manganese ion. The manganese ion may be in a salt form (e.g. manganese chloride). In another aspect, the paramagnetic metal ions used as contrast agents (e.g. manganese) are chelated in order to help avoid toxicity and/or solubility problems. Various manganese chelate image enhancement agents are known: e.g. MnDPDP, MnDTPA, MnEDTA and derivatives, Mn porphyrins such as MnTPPS₄, and fatty acyl DTPA derivatives. Various chelation complexes are described in U.S. Pat. No. 6,797,255 entitled “Methods and compositions for enhancing magnetic resonance imaging” and U.S. Pat. No. 5,980,863 entitled “Manganese compositions and methods for MRI,” which are incorporated by reference. In a variation on chelation, Quay (European patent application 308983) has described the use of manganese amino acid coordination complex solutions. Further, the diagnostically effective quantity of Mn++ ion may be combined with a source of Ca++ ion as generally set forth in U.S. Pat. No. 5,980,863, which is incorporated by reference. In addition, contrast agent isotopes, such as 54 Mn, may be used.

The contrast agent may be administered to the patient in any suitable manner at any suitable location, including oral administration. For some MRI imaging, the most preferred mode for administering the contrast agent will be through parenteral, for example intravenous, administration. A preferred route for manganese involves intracortical administration. The contrast agent is theoretically taken up by the cell body and then transported axonally towards the cortical spinal tract and spinal cord. In addition, the contrast agent may be delivered near the area of the cortical spinal tract or spinal cord to be imaged. For example, it has been shown that administering manganese directly into the spinal cord intrathecally results in uptake by the neurons. See Bilgen, Manganese-enhanced MRI of rat spinal cord injury, Magn. Reson. Imaging. September 23(7):829-32 (September 2005).

Increased uptake of the contrast agent in neurons in areas such as the spinal cord and the brain is preferably performed by applying electrical stimulation to the subject. The electrical simulation causes voltage gated calcium channels in neuronal tissue, organ or system to open, which in turn improves uptake of the contrast agent by the neuron and results in improved manganese-enhanced MRI.

The electrical stimulation used to increase contrast agent uptake may be performed using various types of stimulation devices and electrodes. For example, holes may be made in the subject so that the electrodes rest on the dura. As another example, electrical stimulation with transcutaneous or percutaneous electrodes is contemplated. Transcranial electrical simulation, including acupuncture, is also an established non-invasive method for neuronal stimulation in humans.

The electrodes are preferably placed to stimulate the neurons associated with the cortical spinal tract (both lateral and anterior) and spinal cord. For example, neurons in the cerebral cortex descend directly into the spinal cord to synapse on motor neurons in the anterior gray horns of the spinal cord. Thus, electrical stimulation of the cerebral cortex results in increased contrast agent uptake, which is axonally transported into the cortical spinal cord.

Various patterns of electrical stimulation can be applied in order to stimulate and improve contrast agent uptake by the neuron. For example, the electrical stimulation can be monophasic or biphasic. Typically the pulse duration is about 0.2 ms, although biphasic pulse patterns are preferable because the charge cancellation limits potential tissue damage. The stimulus patterns are chosen to produce substantial increases in the activity of the target neurons. Frequencies generally range from 50 to 500 hz at currents of 0.1 to 5 mA, depending upon the type of electrode employed. The electrical stimulation is usually applied for several minutes, e.g. about 30 to 90 minutes, but is non-continuous (e.g. on 30 seconds and off for about 30 seconds) in order to prevent muscle fatigue. Thus, the stimulus cycle of 25 to 50% on time applied over a period of 30-90 minutes is effective.

In another embodiment, the present invention is directed to improving neuronal contrast agent uptake, such as that in manganese-enhanced MRI, using other techniques for opening neuronal calcium channels. In one aspect, calcium channels may be activated (i.e. opened) using calcium channel agonists, which in turn results in increased influx of manganese into the cell body. Exemplary calcium channel agonists include CGP 28392, Bay K 8644, FPL-64176 (FPL), and maitotox ion.

In another embodiment, the present invention is directed to improving manganese-enhanced MRI by activating calcium channels through indirect calcium channel opening pathways. As an example of an indirect activation, it has been shown that vasopressin receptor activation regulates the influx of extracellular calcium via L-type calcium channel activation through a protein kinase-C-dependent mechanism. See Son et al., Regulation and Mechanism of L-Type Calcium Channel Activation via V1a Vasopressin Receptor Activation in Cultured Cortical Neurons, Neurobiology of Learning and Memory, Vol. 76 No. 3, pp. 388-402(15) (November 2001); see also Dziema, PACAP Potentiates L-Type Calcium Channel Conductance in Suprachiasmatic Nucleus Neurons by Activating the MAPK Pathway, The Journal of Neurophysiology Vol. 88 No. 3 pp. 1374-1386 September 2002; Halling et al., Regulation of voltage-gated Ca2+ channels by calmodulin, Sci STKE. (318):er1 Review (Jan. 17 2006). Thus, administration of therapeutic agents that indirectly open calcium channels may be used to improve the manganese contrast in manganese-enhanced MRI.

The present invention is further illustrated by the following examples, which are merely for the purpose of illustration and are not to be regarded as limiting the scope of the invention or manner in which it may be practiced.

### EXAMPLE 1

### Electrical Stimulation Improves Cortical Spinal Tract Tracing of Spinal Cord Using Manganese Enhanced MRI

In this example, electrical stimulation was applied to the rat cortex in order to improve visualization of the rat spinal cord using manganese-enhanced MRI. The experiments were conducted on twelve Sprague-Dawley rats weighing between 300 and 350 g under a protocol approved by the University of Kansas Medical Center Institutional Animal Care and Use Committee. Six rats were studied without electrical stimulation in the cortex and served as the control group. The remaining six rats formed the stimulation group. These rats received electrical stimulation of motor cortex to test the merits of this procedure as a means of enhancing the delineation of the cortical spinal tract in the spinal cord using manganese-enhanced magnetic resonance imaging.

Surgical Procedures

Rats were anesthetized using ketamine hydrochloride delivered intramuscularly. The advantage of ketamine is that it leaves the excitability of the cortical motor system intact. See Liang et al., Modulation of sustained electromyographic activity by single intracortical microstimuli: comparison of two forelimb motor cortical areas of the rat, Somatosens Mot Res. 10(1):51-61 (1993). The initial dose of ketamine was about 150 mg/kg. This was followed by additional injections at doses of about 5-20 mg/kg, as needed. The head of the anesthetized rat was fixed in a stereotaxic frame (Kopf Instruments, Tujunga, Calif.). A midline incision was made on the scalp from approximately 2.5 mm rostral to about 7.5 mm caudal to the bregma, and the skin was retracted with hemostats. For focal delivery of manganese into the motor cortex, two 1.0 mm burr holes were drilled into the skull bilaterally at 1.5 mm rostral to the bregma and 2.0 mm lateral to the midline using a 1 mm diameter trephine bit attached to a dental drill. Rats in the stimulation group were subjected to an additional craniotomy performed on one side of the skull at a location 6.5 mm caudal to the bregma and 2.0 mm lateral to the midline. Through this opening, a 2.0 mm titanium screw was inserted until it rested on the dura. This screw served as the reference electrode for the electrical stimulation.

Manganese Delivery

A solution containing 1 M manganese chloride (MnCl₂) was prepared and delivered to the rats in both control and study groups through a tapered, graduated micropipette using a 1 μL Hamilton syringe. The syringe was mounted on a micropositioner attached to a stereotaxic frame. The tip of the glass pipette was lowered perpendicular to the brain surface at the center of the first burr hole and inserted into the cortex through the exposed dura one millimeter below the pial surface. The syringe was emptied slowly to deliver 0.2 μL of the solution over a period of five minutes. To prevent backflow of solution, the pipette was left in place for another five minutes prior to withdrawal. This procedure was repeated for the contralateral motor area through the second burr hole.

Cortical Stimulation

Cerebrospinal fluid had a tendency to leak through the punctured dura and to accumulate in the burr hole resulting in shunting of the electrical current. To eliminate this problem, the burr holes in the skull of the rat from the stimulation group were first carefully drained of fluid. Electrical stimulation was then applied through a 1 mm diameter stainless steel electrode with the titanium screw serving as reference. The electrode was connected to a DS7 Digitimer constant current stimulator (Digitimer Ltd., Hertfordshire, England) and lowered through the first burr hole on the same side as the screw until it touched to dura. A Grass S48 stimulator (Grass Medical Instruments, Quincy, Mass.) generated the stimulus pulse sequence used to externally trigger the constant current stimulator. Trains of biphasic stimuli (each phase 0.2 ms, negative first) were applied at 100 Hz for an “on” period of 5 seconds followed by an “off” period of 5 seconds. The threshold for evoking visible motor responses in the forelimb, hindlimb, or tail ranged from 1.1 mA to 1.4 mA. Once threshold was determined, stimulation was applied at approximately twice the threshold for 90 minutes. If the strength of the evoked movement declined noticeably, frequency and current values were increased to maintain a constant motor response. These procedures were repeated for the opposite cortex through a second burr hole. With these settings, the total stimulation time was 180 minutes, 90 minutes for each side of the cortex. Immediately after stimulation, the electrodes were removed, and the skin was closed tightly with suture. The animal was then left to recover in its cage.

Magnetic Resonance Imaging

MRI scans were performed starting as early as about 12 hours after the manganese-delivery and/or electrical stimulation. The animals were anesthetized using spontaneous inhalation of 4% isoflurane for induction, followed by a mixture of 1.5% isoflurane, 30% oxygen, and air delivered through a nose mask. The rat's head was stabilized on a Plexigas holder and positioned into a 6 cm inner diameter volume coil for MRI scanning on a 9.4 T horizontal Varian scanner (Varian Inc., Palo Alto, Calif.). While in the scanner, the physiological condition of the rat was monitored using ECG, respiratory and temperature probes that were connected to an MR-compatible small animal monitoring and gating system (Model 1025, SA Instruments, Inc., Stony Brook, N.Y.). The rat's temperature was kept at about 37° C. by circulating warm air with 40% humidity using a 5 cm diameter plastic tube fitted to the back door of the magnet bore.

After confirming the placement of the animal in the magnet's isocenter with scout images, T₁-weighted volumetric images covering the brain and spinal cord at the cervical and thoracic levels were acquired using a 3-D gradient echo sequence with the parameter values T_{R}/T_{E}=45/4 ms and flip angle (FA)=45°. The data were sampled on a matrix=128×128×64 ranging over a volume of 65×32×28 mm³, and processed and interpolated to 256×256×128 pixels for the final display. Maximum intensity projections were generated to delineate the manganese enhancement over a desired thickness in the coronal and sagittal planes.

Since manganese exerts its effects by changing the magnetic resonance properties of the tissue where it resides, additional scans were performed to characterize the distribution of the longitudinal relaxation time T₁ of the underlying spinal cord and manganese loaded cortical spinal tract. The resulting data set consisted of axial images acquired independently at eight different T_{R} values: 50, 100, 250, 500, 750, 2000, 3500, and 6000 ms with averages=10, 6, 6, 2, 2, 2, 2 and 2, respectively, and T_{E}=12 ms. The other acquisition parameters were image matrix=128×128, field-of-view (FOV)=23×23 mm² and slice thickness=4 mm. The acquired data were analyzed quantitatively to produce a parametric T₁ map of the spinal cord by following the procedures described previously in Bilgen et al., Ex vivo magnetic resonance imaging of rat spinal cord at 9.4 T, Magn. Reson. Imaging 23:601-605 (2005).

Results

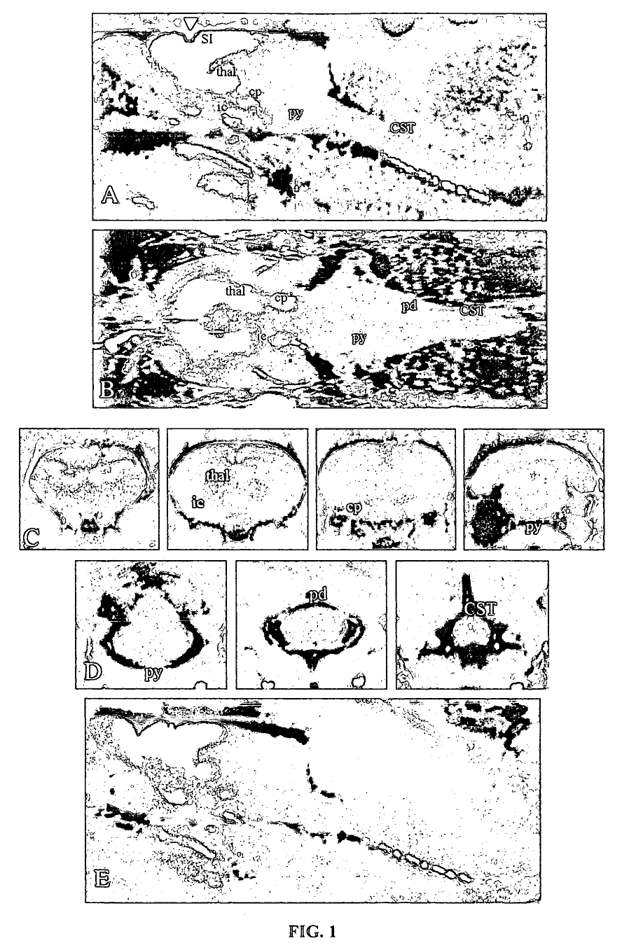

In this example, intracortically administering manganese into the motor areas of the brain and stimulating the cortex electrically provided improved manganese-enhanced MRI. Allowing the rats to rest before the MRI scans helped the animals recover from the surgery and also stabilized the manganese uptake in the cortical spinal tract. FIG. 1 shows manganese enhancement in the brain and spinal cord in views with different orientations. Images in FIG. 1 panels (A) through (D) were obtained from a rat that received electrical stimulation. These images can be compared to the image in FIG. 1 panel (E) from a control rat that received no electrical stimulation. The sagittal images in FIG. 1 panels (A) and (E) were reconstructed from the acquired 3-D data over a thickness of 4 mm using maximum intensity projections and the coronal image in FIG. 1 panel (B) was reconstructed over 8 mm thickness. The images in FIG. 1 panels (C) and (D) represent single slice manganese enhanced MRIs in different axial planes. In both the stimulated and non-stimulated rats, the injection site and the surrounding cortex appear hyperintense due to the presence of highly concentrated manganese. A distinct branch of contrast enhancement breaks away from this region and descends into the subcortical regions of the rat's central nervous system, anterogradely delineating the spatial course of the cortical spinal tract. The anatomical organization of the cortical spinal tract pathway in rat has been described before, using histological techniques. See Donatelle, Growth of the cortical spinal tract and the development of placing reactions in the postnatal rat, J. Comp. Neurol.; 175:207-231 (1977); Paxinos G., The Rat Nervous System, Academic Press: London (1995). The path includes the internal capsule, cerebral peduncle, longitudinal pontine fascicules, pyramid, pyramidal decussation, and the dorsal fascicles of the spinal cord. Manganese enhanced MRIs of stimulated rat clearly depict these neuronal structures and the tracts connected in between with robust and detectable contrast as well as the crossing of the cortical spinal tract at the pyramidal decussation. While the cortical spinal tract runs ventrally near the midline of the medulla before reaching the pyramidal decussation (FIG. 1 panels (A) and (C)), it changes its course at this junction by crossing to the opposite side and descending in the dorsal fasciculus of the spinal cord (FIG. 1 panels (B) and (D)). In the non-stimulated rat, however, manganese enhancement fades away as the cortical spinal tract approaches the spinal cord (FIG. 1(E)).

The ability of the manganese enhanced MRI technique to trace the cortical spinal tract in rat brain has been established by others (Allegrini and Wiessner, 2003; Leergaard et al., 2003). But in these previous studies, MnCl₂ was injected only into one side of the cortex at a single dose 0.8 M in a 10 nL volume (Leergaard et al., 2003) and 1 M in a 1 μL volume (Allegrini and Wiessner, 2003). Using these injection protocols, the reported manganese enhanced MRIs obtained at 24 hours showed only weak labeling of the cortical spinal tract, similar to the one shown in FIG. 1 panel (E). In contrast, the present example employed a comparable concentration (1 M of MnCl₂) but implemented bilateral delivery of 0.2 μL in the cortex, followed by either no electrical stimulation (FIG. 1 panel (E)) or prolonged electrical stimulation (FIG. 1 panels (A)-(E)) at the site of injection. Bilateral injection of manganese has the advantage of producing stronger labeling of the cortical spinal tract, especially in the spinal cord. Although, the data show the feasibility of tracing cortical spinal tract using manganese alone, the addition of electrical stimulation of the cortex yields more robust labeling of the cortical spinal tract in the spinal cord. The data suggest that electrical stimulation enhances the sensitivity and specificity of the manganese approach for labeling the cortical spinal tract not only in brain but also in the spinal cord. This enhanced labeling is most likely a consequence of a stimulation-induced increase in activity of corticospinal neurons leading to increased uptake and anterograde transport of manganese.

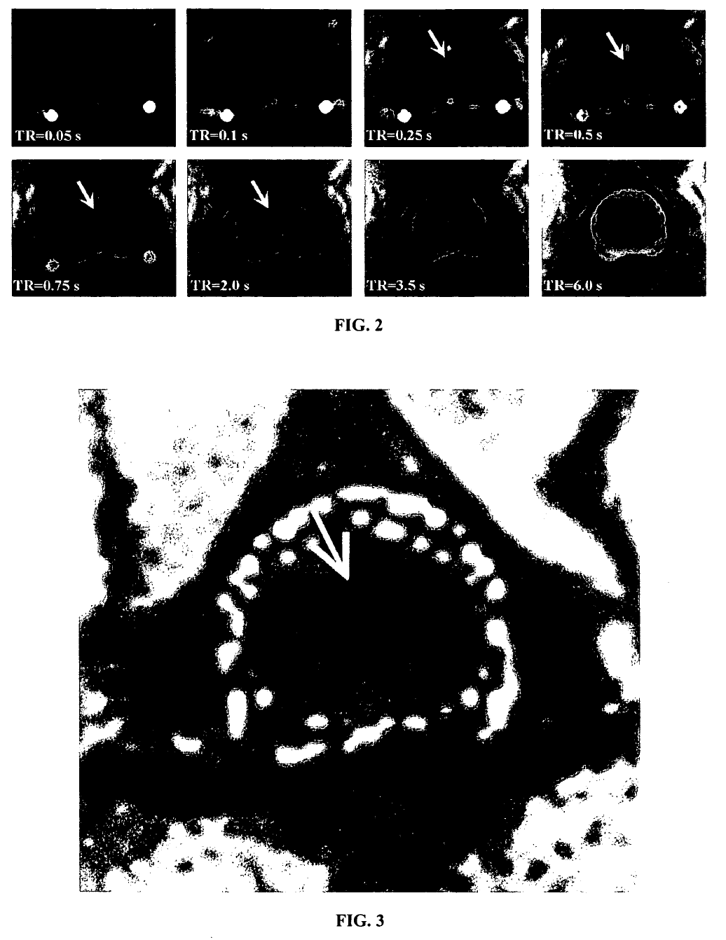

Visualization of the cortical spinal tract in the data was facilitated by contrast enhancement produced by shortening of the T₁ properties of the manganese loaded axonal white matter in cortical spinal tract. To explore the range of T₁-change, the spin-echo images in FIG. 2 were acquired at the cervical level from the spinal cord of a rat that was electrically stimulated. These data exhibit low contrast between the gray matter and white matter on noisy images at low T_{R}, but signal strength and contrast both improve with increased T_{R} due to the proton density and T₁ properties of the spinal cord tissue. See Bilgen et al., Ex vivo magnetic resonance imaging of rat spinal cord at 9.4 T, Magn. Reson. Imaging, 23:601-605 (2005). Processing the images in FIG. 2 produced the T₁-map shown in FIG. 3. This map depicts nearly equal values for T₁ in gray matter and white matter, but a distinct region of hypointensity can be seen as confined to the ventral-most part of the dorsal funiculus of the spinal cord, where the cortical spinal tract is known to lie anatomically (Donatelle, 1977). The spinal cord in rats that were not stimulated electrically did not show this amount of contrast variation over the cortical spinal tract on the T₁-map.

T₁-maps were produced for four rats from each of the stimulated and control groups. Three regions of interest on each map were drawn, and the T₁ values in the gray matter, white matter, and cortical spinal tract were measured, and the mean estimates for each region were calculated separately. Statistics (mean±standard deviation) were computed to take into account the intra-subject variability within each group and for each region. The results from this quantification are given in the table below.

| | Stimulated | Control |
|---|---|---|
| T_{1GM} (s) | 1.54 ± 0.06 | 1.55 ± 0.05 |
| T_{1WM} (s) | 1.51 ± 0.07 | 1.53 ± 0.06 |
| *T_{1CST} (s) | 1.24 ± 0.08 | 1.43 ± 0.09 |

T₁ in the cortical spinal tract of electrically stimulated rats is shorter by about 20%, compared to that of controls. In contrast, T₁ in the gray matter and white matter are nearly the same in both the stimulated and non-stimulated groups. As a corollary, this also implies that manganese is confined to the cortical spinal tract, i.e. remained intra-axonal instead of diffusing into the extracellular spaces.

Signal enhancement in the cortical spinal tract of the thoracic spinal cord as early as about 12 hours following the manganese injection and electrical stimulation was also observed. Considering that the distance from the manganese injection site to the thoracic spinal cord is about 5 cm, the overall transport rate of manganese in cortical spinal tract can be roughly computed as 4 mm/h (i.e., 5 cm/12 hr). Prior researchers have stated that it is difficult to obtain definitive estimates for axoplasmic transport rates, but these researchers give 2.1-2.6 mm/h for cortical and subcortical regions, and 4.6-6.1 mm/h for descending corticofugal pathways (between the internal capsule and the pyramidal decussation) (Leergaard et al., 2003). These rates correspond to relatively fast axonal transport. The data in this example falls within this range and suggests that manganese movement along the cortical spinal tract is due to fast axoplasmic transport rather than diffusion through the tissue.

Examining the data in FIG. 2 more closely also reveals a slight hyperintensity specifically overlaying the cortical spinal tract in the spinal cord images acquired at T_{R}=0.25, 0.5, 0.75, and 2.0. However, the signal enhancement in these spin echo images is not as easily recognizable as the highly localized point-like manganese enhancement seen in the gradient echo image of FIG. 1 panel (D). This is why the protocol of the present invention and the protocol used by others at 3 T (Leergaard et al., 2003) and 4.7 T (Allegrini and Wiessner, 2003) have employed a gradient echo sequence rather than a spin echo sequence to trace the cortical spinal tract.

### EXAMPLE 2

### Electrical Stimulation Improves Imaging of Injured Spinal Cord Using Manganese-Enhanced MRI

Understanding the temporal changes in CNS tissue, such as reorganization of the cortical spinal tract, is an important and growing focus of spinal cord injury (“SCI”) research. In the example, manganese enhanced MRI with electrical stimulation was expanded to include tracing intact neuronal fibers, not only in the cortical spinal tract but also fibers in other tracts, that project through the site of injury.

The experiments were conducted on one Sprague-Dawley rat (about 300 g) under a protocol approved by the University of Kansas Medical Center Institutional Animal Care and Use Committee. Established procedures described previously for the surgery, SCI, manganese delivery and manganese enhanced MRI scans were followed. See Bilgen, A new device for experimental modeling of central nervous system injuries, Neurorehabil. Neural. Repair, 19-226 (2005); Bilgen et al., Ex vivo magnetic resonance imaging of rat spinal cord at 9.4 T, Magn. Reson. Imaging, 23(4):601-605 (2005); U.S. Patent Application Ser. No. 60/756,896 entitled Inductively-Overcoupled Coil Design for High Resolution Magnetic Resonance Imaging. After receiving SCI, the rat was left to recover for two weeks. On post-injury day 14, manganese was delivered intracortically and the motor cortex was stimulated electrically. The next day, manganese enhanced MRI scans were performed on live animals to confirm that the cortical spinal tract labeling in the spinal cord was successful, and on excised cords to obtain high resolution manganese enhanced MRI data around the lesion.

Spinal Cord Injury (“SCI”)

SCI was induced at the T4 level by following the injury protocol described in Bilgen, A new device for experimental modeling of central nervous system injuries, Neurorehabil. Neural. Repair, 19(3):219-226 (2005). Briefly, the rat was anesthetized using spontaneous inhalation of 4% isoflurane for induction and maintained with a mixture of 1.5% isoflurane, 30% oxygen, and air delivered through a nose mask. A rectangular area was shaved on the back and an incision was made to expose the posterior elements of the spine. Then, a rongeur was used to perform a laminectomy at T4 to expose the spinal cord, but to leave the dura intact. After stabilizing the spinal cord with two forceps attached to the rostral T3 and caudal T5 vertebral bodies, the laminectomized section was positioned under the impactor tip of the injury device. Contusion injury was produced by using a rectangular (1 mm×2 mm) injury tip with velocity 1.5 m/s and duration 80 ms. The deformation depth was set to 0.5 mm for producing mild partial injury with good prognosis for behavioral improvement. Next, the skin was closed and the animal was placed in a heated cage to maintain the body temperature while recovering.

Intracortical Mn Delivery

Intracortical delivery of manganese was achieved as described previously. On post injury day 14, the injured rat was anesthetized again using ketamine hydrochloride delivered intramuscularly at an initial dose of 150 mg/kg, followed by additional injections at doses of 5-20 mg/kg, as needed. The head of the anesthetized rat was fixed in a stereotaxic frame (Kopf Instruments, Tujunga, Calif.). A midline incision was made on the scalp from approximately 2.5 mm rostral to 7.5 mm caudal to the bregma, and the skin was retracted with hemostats. Bilateral 1.0 mm diameter burr holes were drilled into the skull 1.5 mm rostral to the bregma and 2.0 mm lateral to the midline using a 1 mm diameter trephine bit attached to a dental drill. An additional craniotomy was performed on one side of the skull at a location 6.5 mm caudal to the bregma and 2.0 mm lateral to the midline. Through this opening, a 2.0 mm diameter titanium screw was inserted until it rested on the dura. This screw served as the reference electrode for the electrical stimulation.

A solution of 1 M MnCl₂ was prepared and filled in a 1 μL Hamilton syringe with a tapered, graduated micropipette tip. A direct stereotaxical injection was made to deliver this solution focally at 0.5 mm below the surface of the cortex through the center of the first burr hole. A total of 0.2 μL of the solution was injected slowly over a period of five minutes. To prevent backflow, the pipette was left in place for another five minutes prior to withdrawal. This procedure was repeated for the contra lateral motor area through the second burr hole.

Cortical Stimulation

Following the manganese delivery, electrical stimulation of the cortex was achieved with a 1 mm diameter stainless steel electrode and the titanium screw serving as reference. The electrode was connected to a DS7 Digitimer constant current stimulator (Digitimer Ltd., Hertfordshire, England) and lowered through the first burr hole on the same side as the screw until it touched to dura. A Grass S48 stimulator (Grass Medical Instruments, Quincy, Mass.) generated the stimulus pulse sequence used to trigger the constant current stimulator. Trains of biphasic stimuli (each phase 0.2 ms, negative first) were applied at 100 Hz for an “on” period of 5 seconds followed by an “off” period of 5 seconds. The current was adjusted until an evoked visible motor response was produced in the forelimb, hindlimb, or tail. The stimulation was applied at approximately twice this threshold for 90 minutes. If the strength of the evoked movement declined noticeably, frequency and current values were increased to maintain a constant motor response. These procedures were then repeated for the opposite cortex through a second burr hole. Immediately after stimulation, the electrodes were removed, and the skin was sutured tightly. The animal was then left to recover in its cage.

Magnetic Resonance Imaging

The cortical spinal tract in rat spinal cord is anatomically located in the ventral-most part of the dorsal funiculus of the SC, i.e., near the central canal between the dorsal horns of the gray matter (GM). Because of this topological arrangement, manganese-labeled cortical spinal tract becomes difficult to differentiate from the gray matter on the. SE image since both structures exhibit similar intensity. The use of 3D gradient-echo (“GE”) sequence with short repetition time however overcomes this limitation and produces robust and detectible manganese-labeled cortical spinal tract in the spinal cord. More recently, inversion recovery spin echo (“IR-SE”) acquisitions were shown to offer better sensitivity to manganese in neuronal tissue. See Tindemans et al., IR-SE and IR-MEMRI allow in vivo visualization of oscine neuroarchitecture including the main forebrain regions of the song control system, NMR Biomed. 19(1):18-29 (2006). Previously, IR-SE imaging was used to demonstrate quantitatively that the T1-relaxation times of the gray matter and white matter are indeed slightly different in the rat spinal cord. See Bilgen et al., Ex vivo magnetic resonance imaging of rat spinal cord at 9.4 T, Magn. Reson. Imaging 23(4):601-605 (2005). Based on the promise that IR-SE provides richer contrast enhancement, IR-SE imaging was also performed to demonstrate its capabilities in visulizing the manganese-labeled cortical spinal tract in addition to the 3D GE imaging.

The MRI scans were performed 24 hours after the manganese-delivery and electrical stimulation. The rat was anesthetized using spontaneous inhalation of 4% isoflurane for induction, followed by a mixture of 1.5% isoflurane, 30% oxygen, and air delivered through a nose mask. The head was stabilized on a Plexiglas holder and positioned in a 6 cm inner diameter volume coil for MRI scanning on a 9.4 T horizontal Varian scanner (Varian Inc., Palo Alto, Calif.). While in the scanner, the physiological condition of the rat was monitored using ECG, respiratory and temperature probes that were connected to an MR-compatible small animal monitoring and gating system (Model 1025, SA Instruments, Inc., Stony Brook, N.Y.). The body temperature was maintained at 37° C. by circulating warm air with 40% humidity using a 5 cm diameter plastic tube fitted to the back door of the magnet bore.

After confirming the placement of the animal in the magnet's isocenter with scout images, T₁-weighted volumetric images covering the brain and spinal cord at the cervical and thoracic levels were acquired using a 3D GE sequence (T_{R}/T_{E}=45/4 ms and flip angle (FA)=45°). The data were sampled on a matrix=128×128×64 ranging over a volume of 70×32×32 mm³, and processed and interpolated to 256×256×128 pixels for the final display. Maximum intensity projections were generated to delineate the manganese enhancement in the cortical spinal tract over a desired thickness in the sagittal plane. Then, a sagittal IR-SE image was acquired (T_{R}/T_{E}/T₁=2000/12/550 ms, field-of-view (FOV)=70×32 mm , image matrix=256×128, slice thickness=2 mm and NEX=4). Finally, axial IR-manganese enhanced MRI was acquired (T_{R}/T_{E}/T₁=2000/17/550 ms, field-of-view (FOV)=22×22 mm², image matrix=128×128, slice thickness=2 mm and NEX=4). The rat was then removed from the scanner, euthanized using cardiac puncture and the vertebral body was dissected from the animal. The excised sample with intact spine was scanned ex vivo at room temperature using an inductively coupled surface coil centered at the injury epicenter described in Bilgen, Simple, low-cost multipurpose RF coilfor MR microscopy at 9.4 T, Magn. Reson. Med. 52(4):937-940 (2004).

High resolution multi-slice SE images were acquired in sagittal and axial planes (sagittal parameters: T_{R}/T_{E}=2500/12 ms, field-of-view=32×10 mm², image matrix=256×128, slice thickness=0.5 mm and NEX=2; axial parameters: T_{R}/T_{E}=2500/12 ms, field-of-view=10×10 mm , image matrix=128×128, slice thickness=2 mm and NEX=2). Finally, axial IR-manganese enhanced MRI of the excised spine and spinal cord were acquired (T_{R}/T_{E}/T₁=2000/15/550 ms, field-of-view (FOV)=10×10 mm², image matrix=128×128, slice thickness=2 mm and NEX=4).

Results

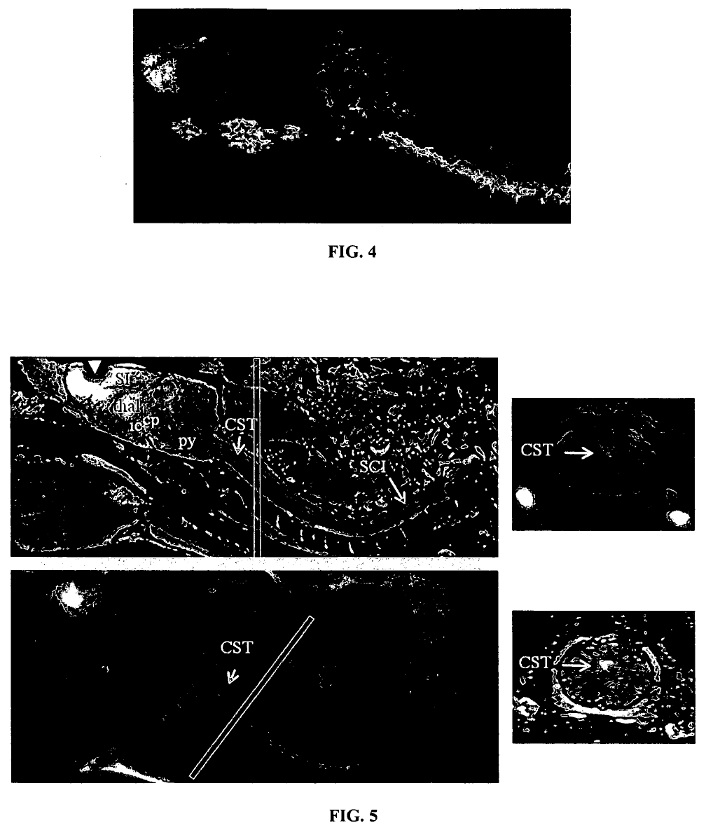

The cortical spinal tract in rat runs caudally from the cortex through the internal capsule, cerebral peduncle, longitudinal pontine fasciculus, pyramid, pyramidal decussation, and descends in the dorsal fasciculus of the spinal cord. FIG. 4 visualizes the spatial projection of this tract in relation to the overall central nervous system by volume rendering the acquired GE-manganese enhanced MRI data in 3D. The manganese-enhancement is represented in red. FIG. 5 depicts the manganese-enhancement in 2D views. The image in FIG. 5 panel (a) shows a single sagittal slice depicting the manganese injection site, the manganese-labeled cortical spinal tract centered within the spinal cord and the spinal cord lesion as delineated by a single patch of signal hypointensity. FIG. 5 panel (b) shows the transverse section of the cervical spinal cord from the imaging plane marked with a thin blue rectangle on the sagittal image. This image shows a nearly circular region of signal hyperintensity located centrally within the spinal cord This bright region with well defined boundary represents the cortical spinal tract labeled with manganese. The corresponding data in FIG. 5 panels (c) and (d) were acquired with IR-manganese enhanced MRI and show the capability of this approach to produce in vivo signal contrast that is also sensitive and specific to the manganese presence in the cortical spinal tract. The lesion was however difficult to visualize on the IR-manganese enhanced MRI in sagittal view. Nevertheless, these volumetric data confirmed that the above experimental procedures were successful in terms of labeling the cortical spinal tract with manganese in live animals.

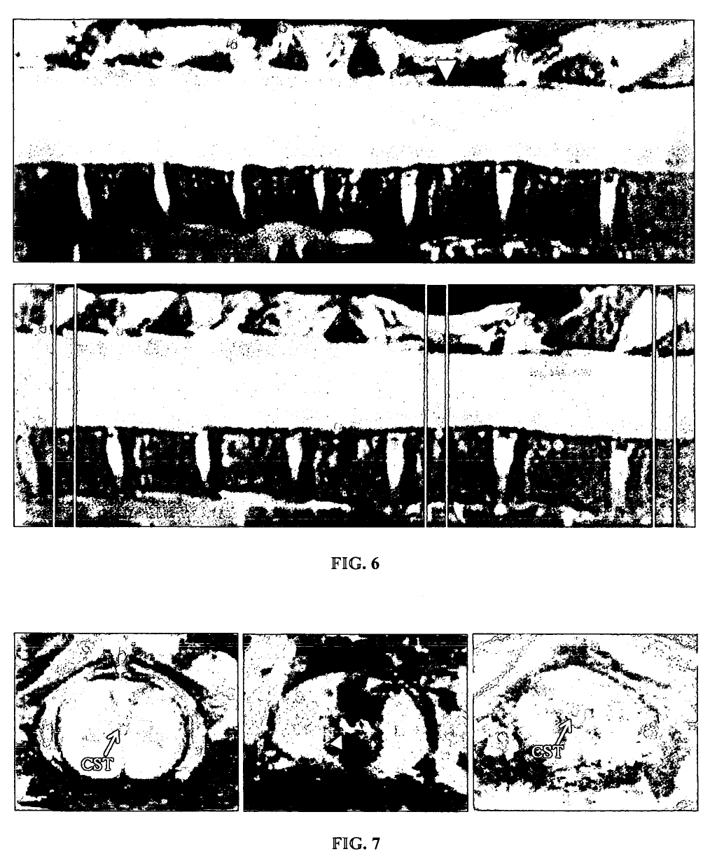

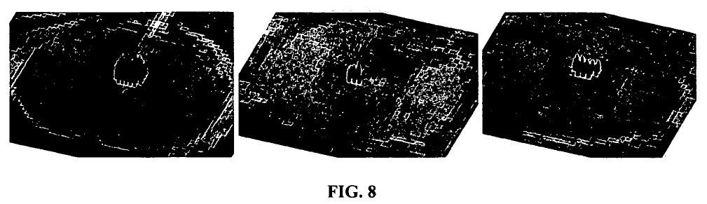

After this confirmation, the experiment was continued with scans on the excised cord to get high resolution data. The resulting ex vivo images from this effort are shown in FIG. 6. The sagittal views in FIG. 6 panels (a) and (b) show the injury and its extent along the spinal cord. The anatomical images from the selected axial planes (blue rectangles in FIG. 6 panel (b)) shown in FIG. 6 panels (c), (d) and (e) depict the injured tissue morphology in greater detail at the epicenter as well as the normal cord at sections rostral and caudal to the injury site. FIG. 7 shows the IR-manganese enhanced MRIs acquired from the same axial slice positions. The gray matter on these images appears relatively darker compared to the white matter in normal sections of the cord. In FIG. 7 panel (a), the pattern of signal enhancement rostral to the injury can be seen as confined spatially to the ventral-most part of the dorsal funiculus between the dorsal horns of the gray matter in sections rostral and caudal the injury. This enhanced region overlap exactly with the expected anatomical location of the cortical spinal tract in the rat spinal cord. Careful examination of the image from the injury epicenter (FIG. 7 panel (b)), a thin strip of signal enhancement, which is situated right posterior to the central canal between the dorsal roots, can be identified. Signal enhancement is also seen in FIG. 7 panel (c) at the expected cortical spinal tract location caudal to the injury. Interpretation of these results requires consideration of possible mechanisms that might facilitate trans-injury propagation of the manganese-dependent contrast. Likely mechanisms include axonal transport and extracellular diffusion of manganese. If the transport were by purely extracellular diffusion, one would expect that the enhancement region would expand gradually from rostral to causal perhaps eventually including the complete cord. However, enhancement was only observed in the location where it would expect to be occupied by the fibers leading from the distal portions of the cord, even in the images obtained caudal to the injury. Since the manganese enhancement remains closely confined to the location of the fibers into which it was originally administered, it is speculated that this indicates a high level of axonal integrity since if the axons are compromised then manganese would be expected to “leak” to the extracellular space and ultimately diffuse away from the enhancing region. Based on these considerations, the manganese-labeling seen at the epicenter slide is consistent with the notion that some cortical spinal tract fibers might have maintained at least some level of connectivity across the injury.

To further support this interpretation, diffusion tensor imaging (“DTI”) was performed on the same slice locations. In previous studies of the rat optic tract, manganese enhanced MRI and DTI have been used as complementary methods to confirm connectivity. Accordingly, it is expected that if the manganese-labeling at the epicenter is truly associated with the underlying connected fibers in the cortical spinal tract, then the water diffusion properties measured within this region would match with those obtained from above and below the lesion. Processing the acquired DTI data pixel-by-pixel produced estimates for the mean water diffusivity, diffusion anisotropy and diffusion direction as respectively represented by the Trace (Tr, average of the diffusion tensor eigenvalues) and fractional anisotropy (“FA”) parameters and principal eigenvector. See Bilgen et al., Mohr diagram interpretation of anisotropic diffusion indices in MRI, Magn. Reson. Imaging 21(5):567-572 (2003). The results from these computations are given in the following table and in FIG. 8.

| | Above the lesion | Epicenter | Below the lesion |
|---|---|---|---|
| Tr (×10⁻³ mm²/s) | 0.69 ± 0.05 | 0.59 ± 0.07 | 0.64 ± 0.06 |
| FA | 0.88 ± 0.08 | 0.79 ± 0.13 | 0.85 ± 0.10 |

The quantitative Tr and FA data in each row of the table as well as the qualitative fiber orientation data in the figure can be seen as all comparing well. The agreement between the Tr values in the table meant that the water diffusivities were similar in the underlying tissues that these measurements were obtained. The agreement between the FA values indicated similar diffusion anisotropy, which indirectly suggested that the underlying tissues had axonal structure. Combining this with the observation that all the principle eigenvectors were oriented along the cord provided alternative evidence that the manganese-labeled tissue at the epicenter was part of the cortical spinal tract.

The use of IR imaging produces tissue contrast variations dependent on the inversion time (T1). In IR acquisitions, the slight differences between the longitudinal relaxation times (T1) in the gray matter and the white matter (including the cortical spinal tract) can be utilized to visualize the gray matter tissue hypointense as compared to the white matter and cortical spinal tract, unlike the image contrast typically seen in the SE images of the spinal cord in FIG. 6. See Bilgen et al., Ex vivo magnetic resonance imaging of rat spinal cord at 9.4 T, Magn. Reson. Imaging 23(4):601-605 (2005). The accumulation of manganese lowers the T1 in cortical spinal tract, introducing a new contrast behavior seen in the IR image of the spinal cord. It was determined that the acquisition parameters provided a good balance between image contrast (enhanced cortical spinal tract versus gray matter and white matter signal suppression) and acquisition time at 9.4 T. Previous in vivo IR studies of the songbird brain at 7 T employed a somewhat different parameter set (T_{R}/T_{E}/T₁=4000/20/855 ms). See Tindemans et al., IR-SE and IR-MEMRI allow in vivo visualization of oscine neuroarchitecture including the main forebrain regions of the song control system, NMR Biomed. 19(1):18-29 (2006). However, variations in the species and organs studied, the experimental protocols followed, and the magnetic field strength may contribute to the differences in imaging parameters.

It is also important to note that high resolution images were obtained using an inductively-coupled surface coil. This coil inherently produces an inhomogeneous rf field that ultimately yields spatially variant inversion. This may be an issue in the IR-manganese enhanced MRI acquisition and lead to an incomplete suppression of the background tissue during the inversion recovery unless the power of the 90° rf pulse is adjusted carefully to focus on the spinal cord.

In sum, this example showed successful imaging of the cortical spinal tract using manganese enhanced MRI and indirectly assessment the axonal fiber connectivity in injured rat spinal cord.

From the foregoing it will be seen that this invention is one well adapted to attain all ends and objectives herein-above set forth, together with the other advantages which are obvious and which are inherent to the invention. Further, since many possible embodiments may be made of the invention without departing from the scope thereof, it is to be understood that all matters herein set forth as shown in the accompanying figures are to be interpreted as illustrative, and not in a limiting sense. Further, it will be understood that certain features and subcombinations are of utility and may be employed without reference to other features and subcombinations. This is contemplated by and is within the scope of the claims.

What is claimed and desired to be secured by Letters Patent is as follows:

1. A method for enhancing imaging of a neuronal tissue, organ, or system in a mammal comprising:
administering a diagnostically effective amount of a contrast agent;
stimulating the mammal's neurons in order to increase uptake of the contrast agent into the neuron.

2. The method of claim 1 wherein said imaging is magnetic resonance imaging.

3. The method of claim 1 wherein said stimulating step comprises applying electrical stimulation to said mammal.

4. The method of claim 3 wherein said electrical stimulation is applied to the motor cortex.

5. The method of claim 3 wherein said electrical simulation is applied in using a biphasic pulse pattern.

6. The method of claim 1 wherein said neuronal tissue that is imaged comprises the mammal's cortical spinal tract or spinal cord.

7. The method of claim 6 wherein said cortical spinal tract or spinal cord has been injured.

8. The method of claim 1 wherein said stimulating step comprises administering a calcium channel agonist to said mammal.

9. The method of claim 1 wherein said contrast agent comprises a paramagnetic metal.

10. The method of claim 9 wherein said paramagnetic metal comprises a source of manganese.

11. The method of claim 10 wherein said source of manganese is a manganese salt.

12. The method of claim 10 wherein said source of manganese is a manganese isotope.

13. The method of claim 10 wherein said source of manganese is administered intracortically.

14. The method of claim 10 wherein said source of manganese is administered intrathecally.

15. A method for enhancing imaging of a neuronal tissue, organ, or system in a mammal comprising:
administering a diagnostically effective amount of a manganese contrast agent;
electrically stimulating the neurons in the cortical spinal tract or spinal cord of said mammal in order to increase uptake of the manganese contrast agent into the neurons.

16. The method of claim 15 wherein said manganese contrast agent is administered to the motor areas of said mammal's brain and said neurons in the cortical spinal tract or spinal cord are electrically stimulated by electrically stimulating the mammal's cortex.

17. The method of claim 15 wherein said manganese contrast agent is a manganese salt.

18. The method of claim 15 wherein said cortical spinal tract or spinal cord has been injured.
